(19) United States
(12) Patent Application Publication
(10) Pub. No.: US 2024/0290453 A1
(43) Pub. Date: Aug. 29, 2024

(54) SUPPLEMENT COMPOUNDING AND PREPARATION TERMINAL, SUPPLEMENT SUPPLY SYSTEM, AND SUPPLEMENT COMPOUNDING AND PREPARATION METHOD

(71) Applicant: KIRIN HOLDINGS KABUSHIKI KAISHA, Tokyo (JP)

(72) Inventors: Hiroyuki HIDA, Tokyo (JP); Keiji YAMANAKA, Tokyo (JP)

(73) Assignee: KIRIN HOLDINGS KABUSHIKI KAISHA, Tokyo (JP)

(21) Appl. No.: 18/693,404

(22) PCT Filed: Sep. 15, 2022
(86) PCT No.: PCT/JP2022/034624

§ 371 Date: Mar. 19, 2024

(30) Foreign Application Priority Data
Sep. 21, 2021 (JP) 2021-153377

(51) Int. Cl. G16H 20/10; G16H 70/40

(52) U.S. Cl. CPC G16H 20/10; G16H 70/40

(57) ABSTRACT

A user terminal includes a transmission/reception unit that acquires first supplement compounding information that indicates compounding of a supplement derived based on a user's information, a display unit that displays the first supplement compounding information acquired by the transmission/reception unit for the user's confirmation, an input reception unit that receives a compounding and preparation input of a supplement that is made by the user regarding the first supplement compounding information displayed by the display unit, a derivation unit that derives second supplement compounding information that indicates compounding of a prepared supplement based on the compounding and preparation input received by the input reception unit, and a transmission/reception unit that outputs the second supplement compounding information derived by the derivation unit.

### TECHNICAL FIELD

One aspect of the present invention relates to a supplement compounding and preparation terminal, a supplement supply system, and a supplement compounding and preparation method.

### BACKGROUND ART

Patent Literature 1 describes a supplement compounding support method in which supplement information that a supply support server should compound is determined in consideration of a user's schedule information, information that can supply a supplement and the like. The use of such a technique makes it possible to propose the determined supplement as a recommended supplement to users.

### CITATION LIST

### Patent Literature

[Patent Literature 1] Japanese Patent No. 6019262

### SUMMARY OF INVENTION

### Technical Problem

Here, proposal of a supplement determined based on a predetermined algorithm is meaningful to users, but it is not easy to appropriately propose a supplement that is highly acceptable to users. As described above, conventionally, there have been cases where it is difficult for users to receive the supply of a supplement suitable for themselves.

One aspect of the present invention has been made in consideration of the above-described circumstances, and an objective of the present invention is to allow users receive the supply of a supplement suitable for themselves.

### Solution to Problem

A supplement compounding and preparation terminal according to one aspect of the present invention includes a first acquisition unit that acquires first supplement compounding information that indicates compounding of a supplement derived based on a user's information, a first display unit that displays the first supplement compounding information acquired by the first acquisition unit for the user's confirmation, an input reception unit that receives a compounding and preparation input of a supplement that is made by the user regarding the first supplement compounding information displayed by the first display unit, a derivation unit that derives second supplement compounding information that indicates compounding of a prepared supplement based on the compounding and preparation input received by the input reception unit, and an output unit that outputs the second supplement compounding information derived by the derivation unit.

In the supplement compounding and preparation terminal according to one aspect of the present invention, the first supplement compounding information derived based on the user's information is displayed, and the compounding and preparation input of a supplement by the user is received. In addition, the second supplement compounding information that indicates the compounding of the prepared supplement based on the received compounding and preparation input is derived, and the second supplement compounding information is output. As described above, the user is allowed to browse the initially proposed first supplement compounding information, the compounding and preparation input by the user is received, and the second supplement compounding information in accordance with the compounding and preparation input is derived and output, whereby compounding information of a supplement in which the user's intention is reflected can be obtained. This makes it possible for the user to receive the supply of a supplement suitable for himself or herself. As described above, according to the supplement compounding and preparation terminal according to one aspect of the present invention, the user can receive the supply of a supplement suitable for himself or herself.

The compounding of the supplement may be at least any one of information regarding a combination of a plurality of supplements, the amount of the supplement, the type of the component of the supplement, the number of the types of the components of the supplement, the compositional ratio of each component of the supplement, the flavor type of the supplement, and the concentration of the supplement. According to such a configuration, the user can tailor the form of a supplement to a form suitable for himself or herself.

The supplement compounding and preparation terminal may further include a second acquisition unit that acquires a code generated based on the second supplement compounding information output by the output unit and a second display unit that displays the code acquired by the second acquisition unit for reading by a supplement server that supplies a supplement. According to such a configuration, it is possible to appropriately and easily transfer the compounding information of a supplement in which the user's intention is reflected to the supplement server through the code and to appropriately and easily perform the supply of a supplement.

A supplement supply system according to one aspect of the present invention is a supplement supply system including a supply support server that supports supply of a supplement and a supplement compounding and preparation terminal that is operated by a user and prepares compounding of the supplement, in which the supply support server derives first supplement compounding information that indicates compounding of a supplement in accordance with the user's information based on a recommendation algorithm that recommends compounding of a supplement according to the user's information and transmits the first supplement compounding information to the supplement compounding and preparation terminal, the supplement compounding and preparation terminal displays the first supplement compounding information for the user's confirmation, receives a compounding and preparation input of a supplement from the user, derives second supplement compounding information that indicates compounding of a prepared supplement based on the compounding and preparation input, and transmits the second supplement compounding information to the supply support server, the supply support server generates a code based on the second supplement compounding information and transmits the code to the supplement compounding and preparation terminal, and the supplement compounding and preparation terminal displays the code for reading by a supplement server that supplies a supplement. According to such a supplement supply system, it is possible to propose compounding of a supplement in accordance with a user's information based on a predetermined recommendation algorithm and, furthermore, to receive a compounding and preparation input by the user to allow the user to receive the supply of a supplement suitable for himself or herself.

The supplement supply system may further include a supplement server that supplies a supplement, and the supplement server may read the code and thereby extract and supply a supplement compounding of which is indicated by the second supplement compounding information. According to such a configuration, it is possible to appropriately and easily transfer the compounding information of a supplement in which the user's intention is reflected to the supplement server through the code and to appropriately and easily perform the supply of a supplement.

A supplement compounding and preparation method according to one aspect of the present invention is a supplement compounding and preparation method that is executed by a supplement compounding and preparation terminal, including a step of acquiring first supplement compounding information that displays compounding of a supplement derived based on a user's information, a step of displaying the first supplement compounding information for the user's confirmation, a step of receiving a compounding and preparation input of a supplement that is made by the user regarding the displayed first supplement compounding information, a step of deriving second supplement compounding information that indicates compounding of a prepared supplement based on the compounding and preparation input, and a step of outputting the second supplement compounding information. According to such a supplement compounding and preparation method, the user can receive the supply of a supplement suitable for himself or herself.

### Advantageous Effects of Invention

According to the supplement supply system according to one aspect of the present invention, users can receive the supply of a supplement suitable for themselves.

### BRIEF DESCRIPTION OF DRAWINGS

FIG. 1 is a view schematically showing a supplement supply system according to a first embodiment of the present invention.

FIG. 2 is a view showing the functional configuration of a supply support server that is included in the supplement supply system of FIG. 1.

FIG. 3 is a hardware configuration view of a control device such as the supply support server.

FIG. 4 is a flowchart of a supply form determination treatment that is executed by the supply support server.

FIG. 5 is a flowchart of an information-for-renewal-use determination treatment that is executed by the supply support server.

FIG. 6 is a view for describing a supplement supply system according to a second embodiment of the present invention.

FIG. 7 is a view showing the functional configuration of a user terminal that is included in the supplement supply system of FIG. 6.

FIG. 8 is a sequence view of a supplement compounding and preparation treatment that is executed by the supplement support server of FIG. 6.

### DESCRIPTION OF EMBODIMENTS

### First Embodiment

Hereinafter, a first embodiment will be described in detail with reference to drawings. In the description, the same element or elements having the same function will be given the same reference sign and will not be described again.

### [Supplement Supply System]

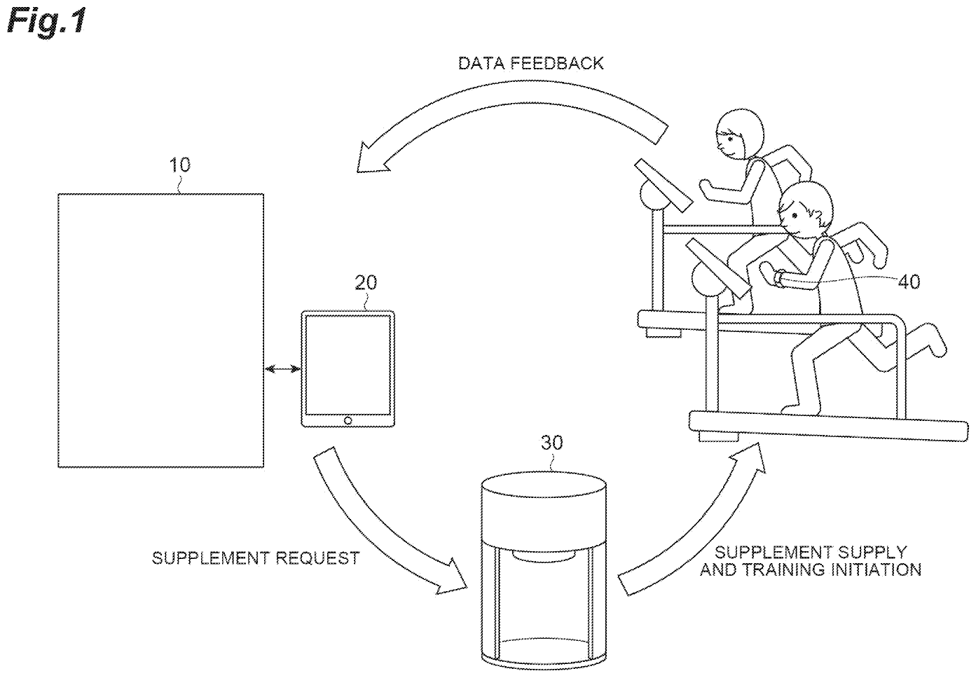

First, the outline of a supplement supply system 1 according to the first embodiment will be described with reference to FIG. 1. FIG. 1 is a view schematically showing the supplement supply system 1 according to the first embodiment. The supplement supply system 1 is a system that supplies a supplement to a user. The supplement supply system 1 determines the supply form of a supplement optimal for each user (tailor-made supplement) and supplies a supplement in a manner that the supply is performed according to the above-described supply form. Here, the supply form refers to a variety of types of information relating to the supply of a supplement such as the type, amount, combination and intake timing of a supplement. In more detail, the supplement supply system 1 supplies a supplement according to a supply form that is determined in consideration of information relating to a user's training. Therefore, “supplement” in the first embodiment refers to a variety of sports supplements, and examples thereof include a variety of proteins, amino acids, conditioning supplements and the like. The form of a supplement to be supplied may be a powdery food or may be a beverage. Hereinafter, an example where the supplement is a powdery food will be described, but the form of the supplement is not limited thereto.

As an example, the supplement supply system 1 is configured to include a supply support server 10, a user terminal 20 (supplement compounding and preparation terminal), a supply server 30 (supplement server), and a wearable device 40 as shown in FIG. 1. In FIG. 1, each configuration of the supplement supply system 1 is drawn singly; however, in practice, a plurality of the user terminals 20 and a plurality of the wearable devices 40 are provided according to the number of users. In addition, the numbers of the supply support servers 10 and the supply servers 30 may also be plural in stead of one. The supply support server 10 is a device that acquires information relating to a user's training and determines the supply form of a supplement in accordance with the information relating to the user's training based on a recommendation algorithm that recommends the supply form of a supplement according to the information relating to the training. The details of the supply support server 10 will be described below.

As the user, users that actively perform training such as athletes can be exemplified, and specific examples thereof include contestants of baseball, soccer, basketball, tennis, rugby, American football, volleyball, table tennis, badminton, a variety of track and field events, skiing, skating, ice hockey and martial arts such as boxing, wrestling, and judo, contestants of competitions for body composition such as bodybuilding and physique and the like.

In addition, examples of the user include people that perform training necessary for each competition three to seven days, four to seven days, five to seven days or six or seven days a week or every day and one to eight hours, two to five hours or three to four hours a day. The user requires training suitable for a competition-oriented schedule to perform high-load training and needs to take in a supplement fitting the training contents to obtain an effective training result also including fatigue recovery within a short period of time.

The user terminal 20 is a communication terminal that is carried by the user, and examples thereof include tablet-type terminals, smartphones, feature phones, notebook-type personal computers and the like. FIG. 1 shows an example where the user terminal 20 is a tablet-type terminal. The user terminal 20 is configured to be capable of wireless communication with the supply support server 10, the supply server 30 and the wearable device 40. The user terminal 20 transmits information that is input by the user, who is an operator, to the supply support server 10. The information that is input by the user is roughly classified into before-training information that is input by the user before training and after-training information that is input (recorded) by the user after training.

The before-training information is, for example, the contents of planned training, the purpose of the planned training, the user's personal data, the user's life data and the like. The contents of the planned training refer to information of, for example, the event, intensity (exercise intensity that is indicated by METS or the like), amount, time and the like of the planned training. The purpose of the planned training refers to information indicating an effect that is required by the user and is, for example, information of muscle strengthening, endurance strengthening, (weight) loss and the like. The user's personal data refers to information of, for example, the user's gender, age, height, weight, athletic career and the like.

For example, in a case where the competition event is bodybuilding, the training event can be bench press, dumbbell bench press (full chest), incline bench press, incline dumbbell bench press (upper chest), decline dumbbell press, dips (lower chest), dumbbell fly, cable crossover (inner chest) or the like when the muscle strengthening area is the pectoral muscle, can be pull-ups (Latissimus dorsi), decline dumbbell rows, one-hand rows, bent-over rows, pullovers, lat pulldowns, low pulley rows (latissimus dorsi), dumbbell rows, shoulder shrugs, deadlifts, bent-over rear raises, knee bent rows, upright rows (trapezius muscle), back extensions, dumbbell good morning (erector muscle), wide grip lat pulldowns, pullovers (teres major muscle), dumbbell rows, dumbbell shrugs, bent-over rows, low pulley rows, T-bar rows or the like when the muscle strengthening area is the back, can be shoulder press (all deltoid muscles), upright rows (all deltoid muscles), front raises (front deltoid muscle), side raises (middle deltoid muscle), rear lateral raises, face pulls, rear delt rows, rear raises (back deltoid muscle) or the like when the muscle strengthening area is a deltoid muscle, can be push-ups, dumbbell triceps press (all triceps), dips, triceps extension, kickbacks, French press (long heads of triceps), press down (short heads of triceps) or the like when the muscle strengthening area is the triceps, can be chin-ups, arm curls (all biceps), concentration curls, incline dumbbell curls (short heads of biceps), hammer curls, side curls (long heads of biceps) or the like when the muscle strengthening area is the biceps, can be ab rollers (rectus abdominis), admiral crunch (rectus abdominis), side bend (obliques), loaded plank (transversus abdominis) or the like when the muscle strengthening area is the abdominal muscle, can be squats, sissy squats, leg presses, leg extensions, hack squats or the like when the muscle strengthening area is the quadriceps femoris muscle, can be squats, sissy squats, leg presses, leg extensions, hack squats or the like when the muscle strengthening area is the hamstrings, can be squats, wide squats, Bulgarian squats, hip abductions, hip extensions, hip lifts, kickbacks, additions or the like when the muscle strengthening area is the gluteal muscles, can be bicycle crunches, leg raises, barbell squats or the like when the muscle strengthening area is the iliopsoas muscle, can be wide squats, side lunges, abduction or the like when the muscle strengthening area is the adductor muscle and can be curl raises or the like when the muscle strengthening area is the triceps surae muscle.

In addition, in a case where the user performs high-intensity training, since the training is performed separately for each strengthening area, the trained area is in the recovery process on the next day of the training or the day after that day, and an effect can be obtained from all muscles by repeating the training on areas other than the above-described area. Therefore, information of not only the training day but also at least seven days from the next day becomes the before-training information.

Furthermore, in a case where a long period of time remains until the competition day, there are cases where the training event, intensity, amount, time and the like are determined on a daily basis in consideration of a several-week schedule, a one-month schedule or the period of time until the competition day. Therefore, information of that period of time also becomes the before-training information.

The after-training information refers to, for example, the contents of the training performed, the information of a supplement that user took in after the training, the user's body sensory information and the like. The contents of the training performed refer to information of, for example, the event, intensity (exercise intensity that is indicated by METS or the like), amount, time and the like of the training performed. The information of a supplement that user took in after the training refers to information of, for example, the type, amount, intake time, purchase price and the like of the supplement taken in. The user's body sensory information refers to a matter (thought) that the user felt regarding the training performed and the supplement taken in and is, specifically, the user's thought, for example, “as a result of taking in an XX supplement after the training, recovery was fast on the next day” or “it was difficult to take in a OO supplement after endurance training.”

The user terminal 20 transmits the information transmitted from the wearable device 40 to the supply support server 10. The wearable device 40 is a terminal that the user wears during training and automatically acquires a variety of types of information such as the heart rate. As the wearable device 40, for example, conventionally well-known wristwatch-type or underwear-type wearable devices can be used. The user terminal 20 acquires log information automatically acquired (recorded) during the training in the wearable device 40 after the training or during the training from the wearable device 40. The wearable device 40 may be a device that directly transmits the above-described log information to the supply support server 10 without going through the user terminal 20.

The user terminal 20 transmits a supplement request to the supply server 30 according to, for example, the user's operation (operation relating to the request of a supplement). When receiving an operation relating to the request of a supplement from the user, the user terminal 20 receives information regarding the supply form of the supplement from the supply support server 10 (or has received the information in advance) and transmits the supplement request to the supply server 30 so that the supplement is supplied according to the supply form of the supplement. The supply server 30 is a server that supplies a supplement according to a supplement request. That is, the supply server 30 stores a plurality of supplements in advance and supplies (discharges) one or a plurality of supplements according to a supplement request. The supply server 30 is installed in, for example, sports gyms or the like. The supply server 30 is a server that supplies a supplement in a supply form determined in the supply support server 10 and supplies supplements according to a type, amount or combination that is indicated by the determined supply form. In addition, in a case where the intake timing of a supplement has been determined in the supply form, the supply support server 10 wraps each supplement in, for example, packaging or the like in which the intake timing is written and supplies each supplement.

### (Supply Support Server)

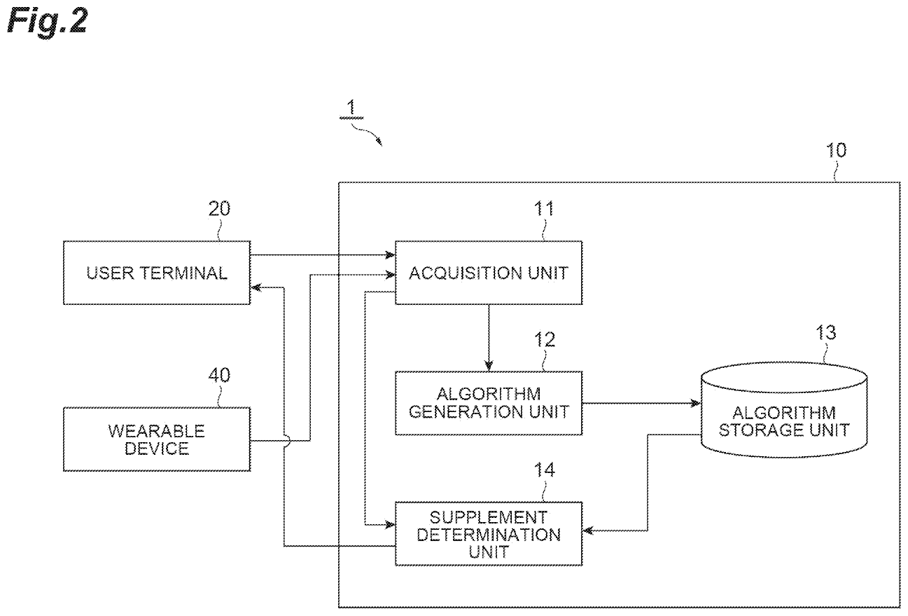

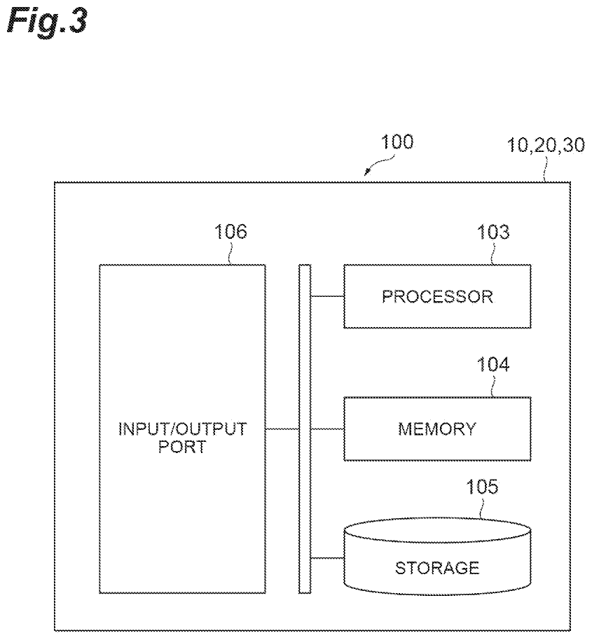

Next, the supply support server 10 will be described with reference to FIG. 2 and FIG. 3. As shown in FIG. 2, the supply support server 10 includes an acquisition unit 11, an algorithm generation unit 12 (generation unit), an algorithm storage unit 13 and a supplement determination unit 14 (determination unit). The supply support server 10 has generated a recommendation algorithm that recommends the supply form of a supplement in advance as information for determining the supply form of a supplement and appropriately renews the recommendation algorithm based on the user's information. In addition, the supply support server 10 inputs information relating to the user's training to the recommendation algorithm and thereby determines a supply form of a supplement in accordance with the information relating to the user's training.

The acquisition unit 11 receives (acquires) the information relating to the user's training from the user terminal 20 and the wearable device 40. Specifically, the acquisition unit 11 acquires training performance information that is obtained by the performance of training by the user from the user terminal 20 and the wearable device 40 as the information relating to the user's training. The acquisition unit 11 acquires the (above-described) after-training information such as the contents of the training performed that is recorded by the user after the training, the information of a supplement taken in by the user after the training and the user's body sensory information from the user terminal 20 as the training performance information. In addition, the acquisition unit 11 acquires the log information that is automatically recorded during the training from the wearable device 40 as the training performance information.

Furthermore, the acquisition unit 11 acquires the (above-described) before-training information that is obtained before the user performs the training from the user terminal 20 as the information relating to the user's training. The acquisition unit 11 acquires the contents of the planned training, the purpose of the planned training, the user's life data and the like as the before-training information.

The algorithm generation unit 12 generates a recommendation algorithm. The recommendation algorithm refers to an algorithm that recommends the supply form of a supplement according to the input information relating to the training. The supply form is a variety of types of information relating to the supplement supply such as the type, amount, combination and intake timing of a supplement. The algorithm generation unit 12 generates the recommendation algorithm based on, for example, information relating to training of a plurality of users and information relating to a supplement that is taken in by the user that is acquired by the acquisition unit 11. In addition, the algorithm generation unit 12 may renew the recommendation algorithm as appropriate based on the information relating to training of a plurality of users and the information relating to a supplement that is taken in by the user. That is, the algorithm generation unit 12 generates and renews the recommendation algorithm in consideration of the correspondence between training and a supplement.

The algorithm generation unit 12 may generate or renew the recommendation algorithm using only information relating to training for users having a training frequency or intensity that is a predetermined threshold value or higher and information relating to a supplement. The threshold value for the training frequency may be set to, for example, three to seven days, four to seven days, five to seven days or six or seven days a week or every day and one to eight hours, two to five hours or three to four hours a day. Regarding the threshold value for the training intensity, the repetition maximum (RM) may be set to 1 to 10 RM when the purpose is, for example, an increase in muscle strength, 10 to 20 RM when the purpose is muscular hypertrophy and 15 to 20 RM when the purpose is muscular endurance improvement. The algorithm generation unit 12 stores the generated recommendation algorithm in the algorithm storage unit 13. In addition, the algorithm generation unit 12 renews the recommendation algorithm that is stored in the algorithm storage unit 13.

The algorithm storage unit 13 stores the recommendation algorithm generated by the algorithm generation unit 12.

The supplement determination unit 14 determines a supply form of a supplement in accordance with the information relating to the user's training acquired by the acquisition unit 11 based on the recommendation algorithm that is stored in the algorithm storage unit 13. The supplement determination unit 14 transmits the determined supply form of a supplement to the user terminal 20. The supplement determination unit 14 may transmit the supply form of a supplement to the user terminal 20 at the time of determination or may transmit the supply form of a supplement to the user terminal 20 according to a request from the user terminal 20.

Specifically, the supplement determination unit 14 inputs the training performance information acquired by the acquisition unit 11 to the recommendation algorithm and thereby determines the supply form of a supplement. That is, the supplement determination unit 14 inputs the log information, the user's body sensory information and the like to the recommendation algorithm and thereby determines the supply form of a supplement.

In addition, the supplement determination unit 14 may determine the supply form of a supplement by inputting the before-training information acquired by the acquisition unit 11 to the recommendation algorithm. That is, the supplement determination unit 14 may determine the supply form of a supplement by inputting the contents of the training, the purpose of the training, the life data and the like to the recommendation algorithm. The supplement determination unit 14 may input some of the above-described training performance information and before-training information to the recommendation algorithm or may input a plurality of types of arbitrary information.

FIG. 3 is a hardware configuration view of the supply support server 10. As described in FIG. 3, the supply support server 10 is configured by a circuit 100 having one or a plurality of processors 103, a memory 104, a storage 105 and an input/output port 106. The input/output port 106 inputs and outputs control signals between the user terminal 20 and the wearable device 40. The storage 105 stores programs for executing a variety of treatments. Any storage may be used as the storage 105 as long as the storage is computer-readable. Specific examples thereof include a hard disk, a non-volatile semiconductor memory, a magnetic disk, an optical disk and the like. The memory 104 temporarily stores a program loaded from the storage 105, a calculation result of the processor 103 and the like. The processor 103 executes a program in cooperation with the memory 104 and thereby configures each of the above-described functional modules.

In the hardware configuration of the supply support server 10, each functional module does not always need to be configured by a program. For example, each functional module of the supply support server 10 may be configured by an exclusive logic circuit or an application specific integrated circuit (ASIC).

The user terminal 20 and the supply server 30 are also, similar to the supply support server 10, configured by a circuit 100 having one or a plurality of processors 103, a memory 104, a storage 105 and an input/output port 106.

### [Supply Form Determination Treatment]

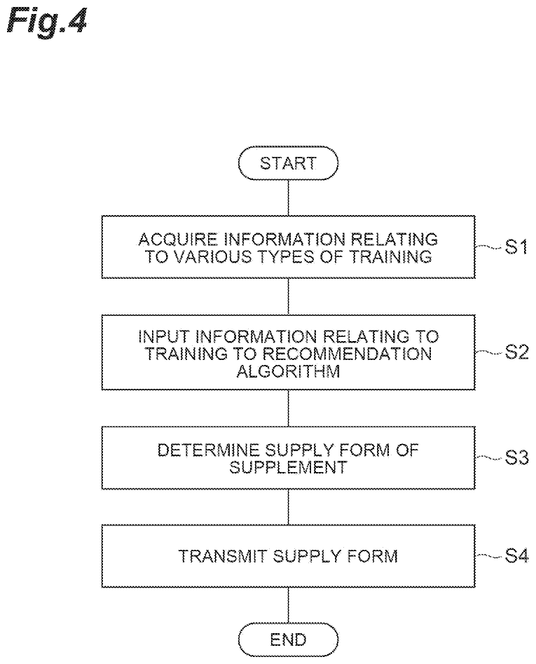

Next, a supply form determination treatment that is executed by the supply support server 10 will be described with reference to FIG. 4. FIG. 4 is a flowchart of the supply form determination treatment that is executed by the supply support server 10. A premise of the treatment is that the recommendation algorithm has been generated in the supply support server 10.

As shown in FIG. 4, the supply support server 10 first acquires information relating to the user's training (step S1). Specifically, the supply support server 10 acquires training performance information that is obtained by the performance of training by the user from the user terminal 20 and the wearable device 40. In addition, the supply support server 10 acquires before-training information that is obtained before the user performs the training from the user terminal 20.

Subsequently, the supply support server 10 inputs the acquired information relating to the user's training to the recommendation algorithm (step S2). In addition, the supply support server 10 determines the supply form of a supplement based on an output result from the recommendation algorithm (step S3). The supply form of a supplement refers to a variety of types of information relating to the supplement supply such as the type, amount, combination and intake timing of a supplement. Finally, the supply support server 10 transmits the determined supply form of a supplement to the user terminal 20 (step S4). What has been described above is the supply form determination treatment that is executed by the supply support server 10. After that, a supplement request is transmitted from the user terminal 20 to the supply server 30, and a supplement in accordance with the supply form is supplied (discharged) to the user from the supply server 30.

### [Information-for-Renewal-Use Determination Treatment]

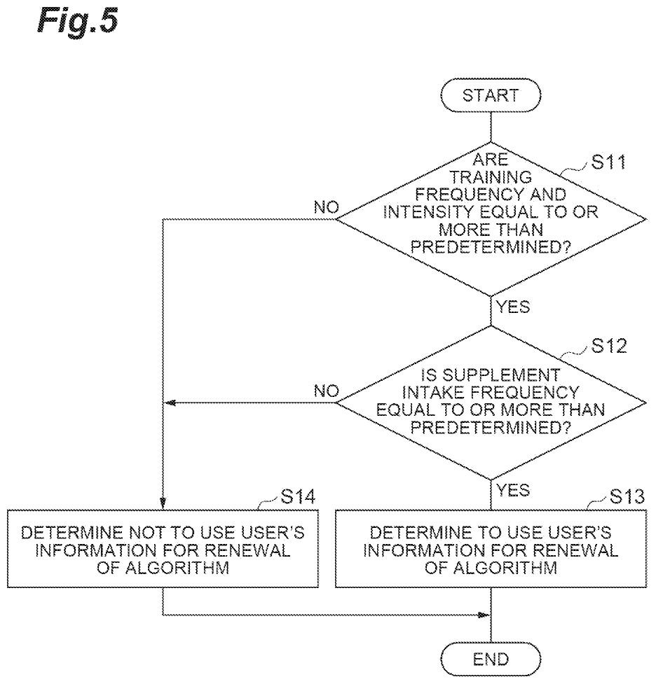

Next, an information-for-renewal-use determination treatment that is executed by the supply support server 10 will be described with reference to FIG. 5. FIG. 5 is a flowchart of the information-for-renewal-use determination treatment that is executed by the supply support server 10. The information-for-renewal-use determination treatment mentioned herein refers to a treatment in which the supply support server 10 determines whether or not the user's information is used for the renewal of the recommendation algorithm.

As shown in FIG. 5, in the information-for-renewal-use determination treatment, first, the supply support server 10 determines whether or not, in the information relating to the user's training that is intended to be used for the renewal of the recommendation algorithm, the training frequency and intensity are predetermined values or more (step S11). In a case where at least any one of the training frequency and intensity is not the predetermined value or more, the information relating to the user's training is determined not to be used for the renewal of the recommendation algorithm (step S14).

On the other hand, in a case where both the training frequency and intensity are the predetermined values or more, the supply support server 10 determines whether or not, in information relating to the user's supplement, the supplement intake frequency is a predetermined value or more (step S12). In a case where the supplement intake frequency is not the predetermined value or more, the information relating to the user's supplement is determined not to be used for the renewal of the recommendation algorithm (step S14). On the other hand, in a case where the supplement intake frequency is the predetermined value or more, the supply support server 10 determines to use the information relating to the user's training and the information relating to the user's supplement for the renewal of the recommendation algorithm (step S13) and performs the renewal of the recommendation algorithm. What has been described above is the information-for-renewal-use determination treatment that is executed by the supply support server 10.

### Action and Effect

Next, the action and effect of the supply support server 10 according to the first embodiment will be described.

The supply support server 10 according to the first embodiment includes the acquisition unit 11 that acquires information relating to the user's training and the supplement determination unit 14 that determines a supply form of a supplement in accordance with the information relating to the user's training based on a recommendation algorithm that recommends a supply form of a supplement according to the information relating to the training.

In the supply support server 10 according to the first embodiment, the information relating to the user's training is input to the recommendation algorithm, and a supply form of a supplement in accordance with the information relating to the user's training is determined. As described above, the information relating to the user's training is taken into account, and, for example, a supply form such as the type, amount, and intake timing of a supplement is determined, whereby a supplement can be supplied in a supply form suitable for the user that performs training.

In the supply support server 10, the acquisition unit 11 acquires training performance information that is obtained by the performance of training by the user as the information relating to the user's training, and the supplement determination unit 14 determines a supply form of a supplement by inputting the training performance information to the recommendation algorithm. As described above, the supply form of a supplement is determined based on information that is obtained by actually performing training, whereby a supplement can be supplied in a supply form corresponding to the training performance result.

In the supply support server 10, the acquisition unit 11 acquires log information that is automatically recorded during the training as the training performance information, and the supplement determination unit 14 determines a supply form of a supplement by inputting the log information to the recommendation algorithm. This makes it possible to supply a supplement corresponding to the training performance result based not on the user's feeling but on the information actually recorded as data.

In the supply support server 10, the acquisition unit 11 acquires the user's body sensory information that is recorded by the user after the training as the training performance information, and the supplement determination unit 14 determines a supply form of a supplement by inputting the user's body sensory information to the recommendation algorithm. This makes it possible to supply a supplement more suitable for the user's desire (feeling) in consideration of the body sensory information such as the thought or the like of the user that has performed the training.

In the supply support server 10, the acquisition unit 11 acquires before-training information that is obtained before the user performs the training as the information relating to the user's training, and the supplement determination unit 14 determines a supply form of a supplement by inputting the before-training information to the recommendation algorithm. The supply form of a supplement is determined based on information that is obtained before the performance of the training, whereby a supplement can be supplied in a supply form corresponding to the training to be performed.

In the supply support server 10, the acquisition unit 11 acquires the contents of planned training as the before-training information, and the supplement determination unit 14 determines a supply form of a supplement by inputting the contents of the training to the recommendation algorithm. This makes it possible to supply a supplement suitable for the contents (the intensity, the amount and the like) of the training to be performed.

In the supply support server 10, the acquisition unit 11 acquires the purpose of planned training as the before-training information, and the supplement determination unit 14 determines a supply form of a supplement by inputting the purpose of the training to the recommendation algorithm. This makes it possible to supply a supplement suitable for the purpose (muscle strengthening, endurance strengthening, weight loss or the like) of the training to be performed.

In the supply support server 10, the acquisition unit 11 acquires the user's life data as the before-training information, and the supplement determination unit 14 determines a supply form of a supplement by inputting the life data to the recommendation algorithm. This makes it possible to supply a supplement suitable for the user's life pattern in consideration of, for example, user-specific life data such as sleeping hours.

The supply support server 10 further includes the algorithm generation unit 12 that generates a recommendation algorithm, and the algorithm generation unit 12 renews the recommendation algorithm based on information relating to training of a plurality of users and information relating to a supplement that is taken in by the user. As described above, the recommendation algorithm is renewed in consideration of the information relating to each user's training and the information relating to a supplement, whereby a supplement having a high relevance to training can be supplied using the recommendation algorithm.

In the supply support server 10, the algorithm generation unit 12 generates or renews the recommendation algorithm using only the information relating to training for users having a training frequency or intensity that is a predetermined threshold value or higher and information relating to a supplement. As described above, the recommendation algorithm is generated (and renewed) using only the information of users having a high training frequency and intensity, whereby it is possible to generate (and renew) the recommendation algorithm without considering, for example, users that do not often perform training (that is, users for which the correspondence between training and a supplement is highly likely to be inappropriate). The use of the recommendation algorithm generated as described above makes it possible to supply a supplement in a supply form more suitable for users that perform training.

In the supply support server 10, the algorithm generation unit 12 generates or renews the recommendation algorithm using only the information relating to training for users having a supplement intake frequency that is a predetermined threshold value or higher and information relating to a supplement. As described above, the recommendation algorithm is generated (and renewed) using only the information of users having a high supplement intake frequency, whereby it is possible to generate (and renew) the recommendation algorithm without considering, for example, users that do not often take in a supplement (that is, users for which the correspondence between training and a supplement is highly likely to be inappropriate). The use of the recommendation algorithm generated as described above makes it possible to supply a supplement in a supply form more suitable for users that perform training.

Hitherto, the supplement supply system 1 according to the first embodiment has been described, but the present invention is not limited to the above-described embodiment. For example, the supplement supply system 1 has been described to have the supply server 30 that supplies (discharges) a supplement, which does not limit the configuration, but may not have the supply server 30. In addition, the supplement supply system 1 may or may not have the wearable device 40. That is, the supplement supply system 1 may not have the wearable device as long as the supplement supply system has the supply support server 10 that determines the supply form of a supplement. For example, in a configuration where the supply server 30 is not provided, the supplement supply system 1 may be configured to send a supplement to users in a supply form determined by the supply support server 10.

In addition, the supply support server 10 has been described to generate and renew the recommendation algorithm, which does not limit the configuration, but may be configured to be incapable of independently generating and renewing the recommendation algorithm and acquire the recommendation algorithm from an external device.

The above-described training frequency and supplement intake frequency will be described in detail.

### Training Intensity

The training intensity can be set according to the purpose of the training. For example, the repetition maximum (RM, the approximate number of times of possible repetition with full power under a given load intensity) is set to 1 to 10 RM in a case where the purpose of the training is to increase the muscle strength of a target muscle (an increase in muscle strength), 10 to 20 RM when the purpose is muscular hypertrophy and 15 to 20 RM when the purpose is muscular endurance improvement. RM and the number of sets are input, whereby the total intensity of the training is calculated, and a supplement in accordance with the intensity is provided. In addition, in a case where it is intended to set the training intensity using calories consumed as an index, it is also possible to use the METs value. For example, since METs have already been determined for a variety of training intensities and for each training event such as 3.5 METs in the case of mild training such as bodyweight abdominal muscle or back muscle exercises, push-ups or squats, 6.0 METs in the case of high-intensity weight training and 10 METs in the case of running at a speed of 9.7 Km/h, when the METs value, the own weight and the training time are input, total calories consumed in the case of the training has been performed is calculated, and a supplement in accordance with the calories is provided.

### Supplement Intake Frequency

The intake of a supplement may be taken in at the same time of training or may be taken in together with meals. Therefore, in a case where a supplement is taken in at the same time as training, the intake frequency is the same as the frequency of the training, and the supplement is taken in three to seven days, four to seven days, five to seven days or six or seven days a week or every day. In a case where a supplement is taken together with training, the supplement is taken in before the training, in the middle of the training or after the training according to the training contents or the type of the supplement. In addition, in a case where a supplement is taken in together with meals, the supplement may be taken in each meal according to the user's meal frequency. The training intake frequency per day can be set to once to 10 times according to the training frequency or the meal frequency.

### Second Embodiment

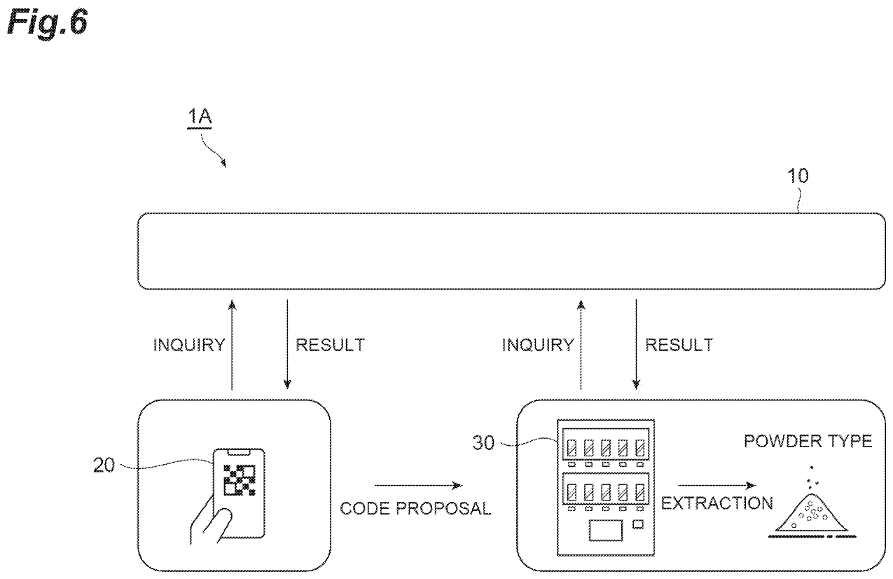

Next, a second embodiment of the present invention will be described with reference to FIG. 6 to FIG. 8. In the second embodiment, differences from the first embodiment will be mainly described, and duplicate descriptions with the first embodiment will not be made.

FIG. 6 is a view for describing a supplement supply system 1A according to the second embodiment. The supplement supply system 1A is a system that supplies a supplement to a user, which is the same as the supplement supply system 1 described in the first embodiment, and, furthermore, has a function of receiving a compounding and preparation input relating to the compounding of a supplement proposed to a user and preparing (correcting) the compounding of the supplement according to the compounding and preparation input. The supplement supply system 1A includes a supply support server 10 that supports the supply of a supplement, a user terminal 20 (supplement compounding and preparation terminal) that is operated by a user and prepares the compounding of the supplement, and a supply server 30 (supplement server) that supplies the supplement.

The supply support server 10 derives first supplement compounding information that indicates the compounding of a supplement in accordance with the user's information based on a recommendation algorithm that recommends the compounding of a supplement in accordance with the user's information. Here, in the first embodiment, the recommendation algorithm is an algorithm that “recommends a supply form of a supplement according to information relating to the user's training”; however, in the present embodiment, the recommendation algorithm may be an algorithm that “recommends the compounding of a supplement according to the user's information.” The user's information is, for example, biological information such as the user's gender, weight, height, age and physical condition, information relating to the user's training, information relating to the user's supplement intake or the like. The information relating to the user's training is, for example, the above-described training performance information that is obtained by the performance of training by the user (the contents of performed training that is recorded by the user after the training, information of a supplement taken in by the user after training, the after-training information such as the user's body sensory information, the log information that is automatically recorded during training), before-training information that is obtained before the user performs the training or the like. The information relating to the user's supplement intake is, for example, the amount, combination, flavor type and the like of a supplement that the user took in in the past. The user's information may be, for example, information containing at least one kind of the above-described information. In addition, the compounding of a supplement is, for example, at least one of information relating to a combination of a plurality of supplements, the amount of the supplement, the type of the component of the supplement, the number of the types of the components of the supplement, the compositional ratio of each component of the supplement, the flavor type of the supplement, and the concentration of the supplement.

The supply support server 10 first acquires the user's information (for example, biological information such as the user's gender, weight, height, age and physical condition, information relating to the user's supplement intake or the like) from the user terminal 20. In addition, the supply support server 10 derives first supplement compounding information that indicates the compounding of a supplement in accordance with the user's information based on a unique recommendation algorithm using the acquired user's information and transmits the derived first supplement compounding information to the user terminal 20.

In addition, the supply support server 10 acquires second supplement compounding information (the details will be described below) that indicates the compounding of the prepared supplement from the user terminal 20, generates a code based on the second supplement compounding information and transmits the code to the user terminal 20. Here, the code refers to, for example, a QR code. In addition, the code is not limited to the QR code and may be a different readable code such as a passcode or a barcode.

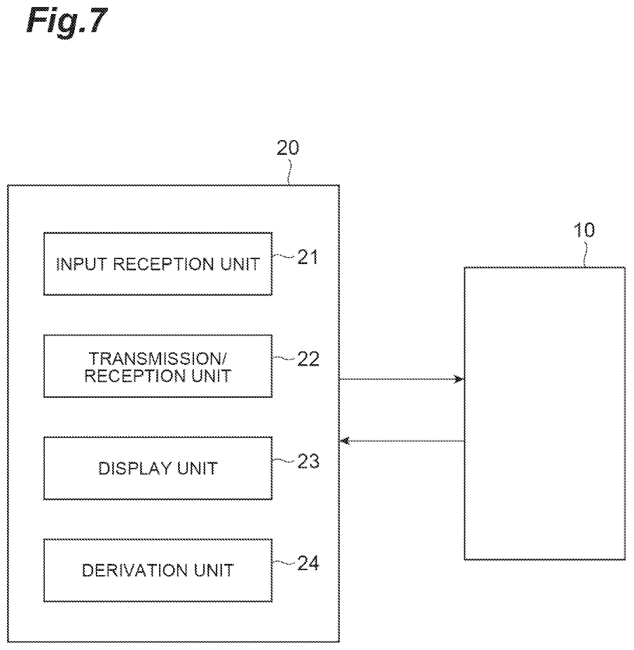

The user terminal 20 is a supplement compounding and preparation terminal and includes, as functional configuration elements, an input reception unit 21, a transmission/reception unit 22 (a first acquisition unit, an output unit and a second acquisition unit), a display unit 23 (a first display unit and a second display unit), and a derivation unit 24 as shown in FIG. 7.

The input reception unit 21 receives an input from the user. The input reception unit 21 receives an input from the user regarding the user's information. That is, the input reception unit 21 receives an input from the user regarding, for example, the user's gender, weight, height and age, the supplement intake timing or the like.

The transmission/reception unit 22 transmits and receives information to and from the supply support server 10. The transmission/reception unit 22 transmits the user's information received by the input reception unit 21 to the supply support server 10. In addition, the transmission/reception unit 22 receives (acquires) the first supplement compounding information derived in the supply support server 10 based on the user's information from the supply support server 10.

The display unit 23 displays a variety of types of information to the user terminal 20. The display unit 23 displays the first supplement compounding information acquired by the transmission/reception unit 22 for the user's confirmation. Such first supplement compounding information is information before a compounding and preparation input by the user is received (before the user's intention is reflected) and is simply a digital value of supplement compounding that is derived from the user's information based on the recommendation algorithm.

The input reception unit 21 receives a compounding and preparation input of a supplement that is made by the user regarding the first supplement compounding information displayed by the display unit 23. The input reception unit 21 receives the user's customized input (information of corrected compounding) regarding, for example, the type, amount (for example, in the unit of grams), flavor type, concentration and the like of a supplement as the compounding and preparation input of a supplement.

The derivation unit 24 derives second supplement compounding information that indicates the compounding of a prepared supplement based on the compounding and preparation input received by the input reception unit 21. A storage unit (not shown) of the user terminal 20 may store the second supplement compounding information (information of corrected compounding) regarding the user so that the second supplement compounding information can be retrieved at the time of next compounding.

The transmission/reception unit 22 transmits (outputs) the second supplement compounding information derived by the derivation unit 24 to the supply support server 10. In addition, the transmission/reception unit 22 receives (acquires) the code generated in the supply support server 10 based on the second supplement compounding information from the supply support server 10. The code that is generated based on the second supplement compounding information may be a QR code or may be a different readable code such as a passcode or a barcode.

The display unit 23 displays the code acquired by the transmission/reception unit 22 for reading by the supply server 30 that supplies a supplement.

The supply server 30 reads the code displayed in the user terminal 20 and thereby extracts and supplies a supplement the compounding of which is indicated by the second supplement compounding information. The supply server 30 makes an inquiry about compounding amount data to the supply support server 10 based on the code reading result. In addition, the supply server 30 receives an answer of the compounding amount data from the supply support server 10 and initiates the ejection of a supplement. The supply server 30 may have, for example, a weight sensor that measures the ejection amount, compare a request amount from the supply support server 10 and the ejection amount measured by the weight sensor and accurately measure whether or not the ejection amount reaches the predetermined request amount (for example, in the unit of grams).

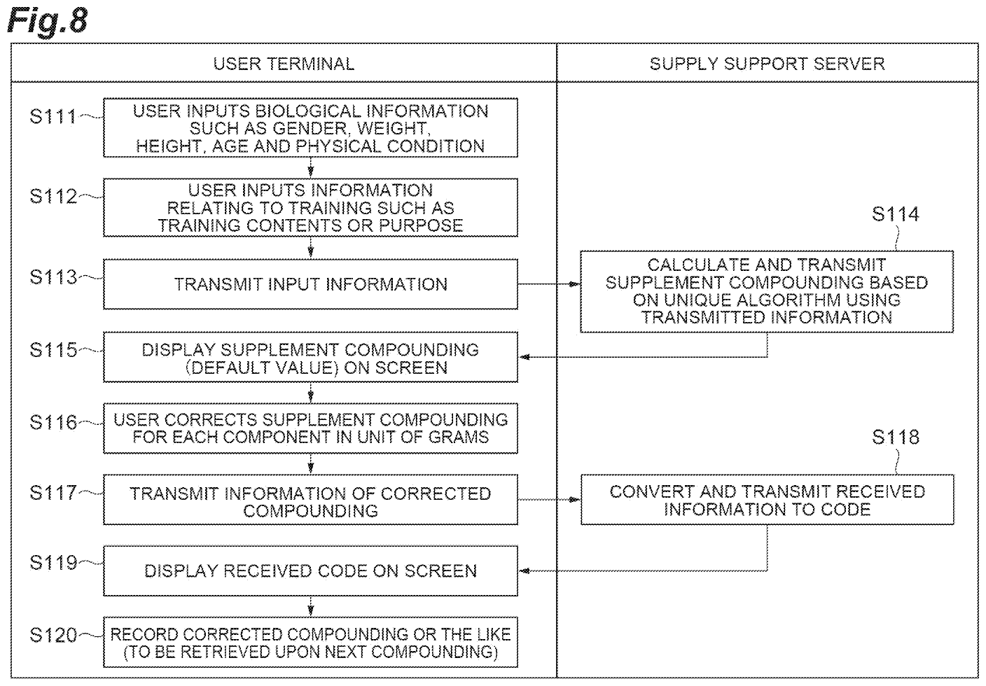

Next, a supplement compounding and preparation treatment that is executed by the supplement supply system 1A will be described with reference to FIG. 8. FIG. 8 is a sequence view of the supplement compounding and preparation treatment that is executed by the supplement supply system 1A.

As shown in FIG. 8, first, an input of biological information such as the gender, the weight, the height, the age and the physical condition by the user is received in the user terminal 20 (step S111).

Subsequently, an input of information regarding training such as the training contents or purpose (information relating to training) by the user is received in the user terminal 20 (step S112). A supplement intake timing refers to, for example, pre-workout, post-workout or the like as described above.

Subsequently, the received information is transmitted from the user terminal 20 to the supply support server 10 (step S113). In addition, in the supply support server 10, first supplement compounding information is derived based on a unique algorithm using the transmitted information, and the first supplement compounding information is transmitted to the user terminal 20 (step S114).

In addition, the first supplement compounding information (digital value) is displayed on the screen in the user terminal 20 (step S115). The user browses the displayed first supplement compounding information and makes a compounding and preparation input of a supplement. That is, in the user terminal 20, for example, regarding the compounding of a supplement, a correction input of each component in the unit of grams and a correction input of the flavor type and the concentration are received (step S116). The user terminal 20 derives second supplement compounding information based on the compounding and preparation input.

Subsequently, the second supplement compounding information, which is the corrected compounding information, is transmitted from the user terminal 20 to the supply support server 10 (step S117). The information received in the supply support server 10 is converted into a code, and the code is transmitted to the user terminal 20 (step S118).

In addition, the code is displayed in the user terminal 20 (step S119), whereby it becomes possible for the second supplement compounding information to be read by the supply server 30. In the end, the second supplement compounding information, which is the corrected compounding information, may be recorded so that the second supplement compounding information can be retrieved at the time of next compounding (step S120).

### Action and Effect

Next, the action and effect of the user terminal 20 and the supplement supply system 1A according to the second embodiment will be described.

The user terminal 20 according to the present embodiment includes the transmission/reception unit 22 that acquires first supplement compounding information that indicates the compounding of a supplement derived based on the user's information, the display unit 23 that displays the first supplement compounding information acquired by the transmission/reception unit 22 for the user's confirmation, the input reception unit 21 that receives a compounding and preparation input of a supplement that is made by the user regarding the first supplement compounding information displayed by the display unit 23, the derivation unit 24 that derives second supplement compounding information that indicates the compounding of a prepared supplement based on the compounding and preparation input received by the input reception unit 21, and the transmission/reception unit 22 that outputs the second supplement compounding information derived by the derivation unit 24.

In such a user terminal 20, the first supplement compounding information derived based on the user's information is displayed and the compounding and preparation input of a supplement made by the user is received. In addition, the second supplement compounding information that indicates the compounding of a prepared supplement based on the received compounding and preparation input is derived, and the second supplement compounding information is output. As described above, the first supplement compounding information proposed in the beginning is browsed by the user, the compounding and preparation input by the user is received, and the second supplement compounding information in accordance with the compounding and preparation input is derived and output, whereby compounding information of a supplement in which the user's intention is reflected can be obtained. This makes the user to receive the supply of a supplement suitable for himself or herself. As described above, according to the user terminal 20, the user can receive the supply of a supplement suitable for himself or herself.

The compounding of a supplement may be at least any one of information regarding a combination of a plurality of supplements, the amount of the supplement, the type of the component of the supplement, the number of the types of the components of the supplement, the compositional ratio of each component of the supplement, the flavor type of the supplement, and the concentration of the supplement. According to such a configuration, the user can tailor the form of a supplement to a form suitable for himself or herself.

The user terminal 20 may acquire a code generated based on the second supplement compounding information and display the code for reading by the supply server 30 that supplies a supplement. According to such a configuration, it is possible to appropriately and easily transfer the compounding information of a supplement in which the user's intention is reflected to the supply server 30 through the code and to appropriately and easily perform the supply of a supplement.

The supplement supply system 1A according to the present embodiment is a supplement supply system including the supply support server 10 that supports the supply of a supplement and the user terminal 20 that is operated by the user and prepares the compounding of a supplement, in which the supply support server 10 derives first supplement compounding information that indicates the compounding of a supplement in accordance with the user's information based on a recommendation algorithm that recommends the compounding of a supplement in accordance with the user's information and transmits the first supplement compounding information to the user terminal 20, the user terminal 20 displays the first supplement compounding information for the user's confirmation, receives a compounding and preparation input of a supplement from the user, derives second supplement compounding information that indicates the compounding of a prepared supplement based on the compounding and preparation input, and transmits the second supplement compounding information to the supply support server 10, the supply support server 10 generates a code based on the second supplement compounding information and transmits the code to the user terminal 20, and the user terminal 20 displays the code for reading by the supply server 30 that supplies a supplement. According to such a supplement supply system 1A, it is possible to propose compounding of a supplement in accordance with the user's information based on the predetermined recommendation algorithm and, furthermore, to receive the compounding and preparation input by the user to allow the user to receive the supply of a supplement suitable for himself or herself.

In the supplement supply system 1A, the supply server 30 may read the code and thereby extract and supply a supplement compounding of which is indicated by the second supplement compounding information. According to such a configuration, it is possible to appropriately and easily transfer the compounding information of a supplement in which the user's intention is reflected to the supply server 30 through the code and to appropriately and easily perform the supply of a supplement.

Hitherto, the second embodiment of the present invention has been described, but the present invention is not limited to the second embodiment. For example, the user terminal 20 has been described as the supplement compounding and preparation terminal, but at least a part of the functions of the supplement compounding and preparation terminal may be performed in the supply support server 10 or the supply server 30.

### REFERENCE SIGNS LIST

1A Supplement supply system
10 Supply support server
20 User terminal (supplement compounding and preparation
terminal)
21 Input reception unit
22 Transmission/reception unit (first acquisition unit, output unit,
second acquisition unit)
23 Display unit (first display unit, second display unit)
24 Derivation unit
30 Supply server (supplement server).

1. A supplement compounding and preparation terminal comprising:
a first acquisition unit configured to acquire first supplement compounding information that indicates compounding of a supplement derived based on a user's information;
a first display unit configured to display the first supplement compounding information acquired by the first acquisition unit for the user's confirmation;
an input reception unit configured to receive a compounding and preparation input of a supplement that is made by the user regarding the first supplement compounding information displayed by the first display unit;
a derivation unit configured to derive second supplement compounding information that indicates compounding of a prepared supplement based on the compounding and preparation input received by the input reception unit; and
an output unit configured to output the second supplement compounding information derived by the derivation unit.

2. The supplement compounding and preparation terminal according to claim 1, wherein the compounding of the supplement is at least any one of information regarding a combination of a plurality of supplements, an amount of the supplement, a type of a component of the supplement, the number of the types of the components of the supplement, a compositional ratio of each component of the supplement, a flavor type of the supplement, and a concentration of the supplement.

3. The supplement compounding and preparation terminal according to claim 1, further comprising:
a second acquisition unit configured to acquire a code generated based on the second supplement compounding information output by the output unit; and
a second display unit configured to display the code acquired by the second acquisition unit for reading by a supplement server that supplies a supplement.

4. A supplement supply system comprising:
a supply support server configured to support supply of a supplement; and
a supplement compounding and preparation terminal configured to be operated by a user and prepares compounding of the supplement,
wherein the supply support server derives first supplement compounding information that indicates compounding of a supplement in accordance with the user's information based on a recommendation algorithm that recommends compounding of a supplement according to the user's information and transmits the first supplement compounding information to the supplement compounding and preparation terminal,
the supplement compounding and preparation terminal displays the first supplement compounding information for the user's confirmation, receives a compounding and preparation input of a supplement from the user, derives second supplement compounding information that indicates compounding of a prepared supplement based on the compounding and preparation input, and transmits the second supplement compounding information to the supply support server,
the supply support server generates a code based on the second supplement compounding information and transmits the code to the supplement compounding and preparation terminal, and
the supplement compounding and preparation terminal displays the code for reading by a supplement server that supplies a supplement.

5. The supplement supply system according to claim 4, further comprising:
a supplement server configured to supply a supplement,
wherein the supplement server reads the code and thereby extracts and supplies a supplement compounding of which is indicated by the second supplement compounding information.

6. A supplement compounding and preparation method that is executed by a supplement compounding and preparation terminal, the method comprising:
a step of acquiring first supplement compounding information that displays compounding of a supplement derived based on a user's information;
a step of displaying the first supplement compounding information for the user's confirmation;
a step of receiving a compounding and preparation input of a supplement that is made by the user regarding the displayed first supplement compounding information;
a step of deriving second supplement compounding information that indicates compounding of a prepared supplement based on the compounding and preparation input; and
a step of outputting the second supplement compounding information.
